(19) United States
(12) Patent Application Publication
(10) Pub. No.: US 2018/0014981 A1
(43) Pub. Date: Jan. 18, 2018

(54) PATIENT FLUID ABSORPTION AND ESTIMATION

(71) Applicant: WASHINGTON STATE UNIVERSITY, Pullman, WA (US)

(72) Inventors: Martin Schiavenato, Colbert, WA (US); Hang Liu, Pullman, WA (US); Subhanshu Gupta, Pullman, WA (US)

(21) Appl. No.: 15/647,125

(22) Filed: Jul. 11, 2017

Related U.S. Application Data
(60) Provisional application No. 62/361,403, filed on Jul. 12, 2016.

(51) Int. Cl. A61F 13/44; A61F 13/42; A61F 13/505; A61F 13/00

(52) U.S. Cl. CPC A61F 13/44; A61F 13/505; A61F 13/42; A61F 2013/423; A61F 2013/424; A61F 2013/0094

(57) ABSTRACT

A patient fluid absorption and estimation device includes an absorbent layer comprising an absorbent material and adapted to absorb fluids. The device's sensing layer includes piezoresistive element(s). The sensing layer abuts the absorbent layer such that changes in at the mass or shape of the absorbent layer cause a change in the strain in the piezoresistive element(s). The device includes memory and processing in communication with the memory. The processing includes a piezoresistive element interface. The processing senses a voltage, representing an estimation of the amount of fluid absorbed, across each element via the interface. The processing then converts each voltage to a digital value, and stores the value in the memory. The device includes wireless communications that receives commands from an external computer system to transmit the values, and transmits the values to the external. A power subsystem powers each subsystem.

### CROSS REFERENCE TO RELATED APPLICATIONS

This application claims the benefit of U.S. Provisional Application No. 62/361,403, entitled “Smart Diaper,” and filed Jul. 12, 2016. The entire contents of the above-identified priority application are hereby fully incorporated herein by reference.

### TECHNICAL FIELD

The technology disclosed herein is related to fluid estimation. Particular embodiments relate to estimating urinary output of a patient using a diaper having integrated piezoresistive elements.

### BACKGROUND

Urinary output (UO), and fluid (such as blood) output generally, is an important metric in the diagnosis, monitoring, and treatment of many disorders including kidney disease, heart failure, dehydration, and shock. Premature infants in the newborn intensive care unit (NICU) undergo routine fluid and electrolyte management requiring close monitoring of input, e.g., intravenous (IV) fluids, and UO. This population is characterized by several traits that make them especially vulnerable to fluid and electrolyte imbalances including a higher percentage of total body water, and an incompletely developed renal system. UO is such a critical element in the care of these infants that it has been identified as a predictor of mortality in the NICU. The current clinical measurement method for UO in the modem NICU involves diaper removal and transport to a digital scale, followed by zeroing the scale with an unused diaper, removing the unused diaper, and then placing the used diaper unto the scale. The clinician then notes the scale output, disposes of the used diaper, and proceeds to document (manually or otherwise) the finding in the record to include output in milliliters (x grams=x milliliters), date, and time. This process is both time consuming and unhygienic, requiring repeated cleaning/disinfecting of common space and equipment; and the costs associated with clinician time and disinfecting chemical products. An infant in the NICU will have their diaper changed every 3-6 hours; that is an average of 4-8 diapers per infant; given a 30 bed NICU, this process is repeated 120-240 times/day.

### SUMMARY

Embodiments of the technology disclosed herein include methods, devices, systems, and computer program products to estimate patient fluid output. In some embodiments, the technology includes a patient fluid absorption and estimation device. The device includes an absorbent layer comprising an absorbent material and adapted to absorb fluids. A sensing layer of the device includes at least one piezoresistive element. The sensing layer abuts the absorbent layer such that changes in at least one of the mass of the absorbent layer and the shape of the absorbent layer cause a change in the strain in the at least one piezoresistive element.

The device includes a memory, and a processing subsystem in communication with the memory. The processing subsystem includes a piezoresistive element interface. The processing subsystem is operative to sense a voltage, representing an estimation of the amount of fluid absorbed in the absorbent layer, across each piezoresistive element via the piezoresistive element interface. The processing system then converts each sensed voltage to a digital value, and stores each digital value in the memory.

The device includes a wireless communications subsystem in communication with the processing subsystem and the memory. The wireless communication subsystem is operative to receive commands from an external computer system to transmit the stored values, and operative to transmit the stored values to the external computer system in response to the commands. A power subsystem is in electrical communication with the processing subsystem and the communications subsystem, and operative to power each subsystem.

In some embodiments, the technology is a system including the device described above, along with a computer program product that is executable on the external computer system. The computer program product includes a non-transitory computer-readable storage device having computer-executable program instructions embodied thereon that when executed by the external computer system, cause the external computer system to interface with the fluid absorption and estimation device. The instructions cause the external computer system to command the fluid absorption and estimation device to transmit the stored values to the external computer system; to receive, in response to the command, and from the fluid absorption and estimation device, the transmitted values; and to convert the received values to an estimate of fluid absorbed by the fluid absorption and estimation device.

In some system embodiments, the computer-executable program instructions further cause the external computer system to transmit the converted values from the external computer system to a medical records system, as an estimate of fluid absorbed by the device.

In some embodiments of both the system and the device, the power subsystem comprises an ambient harvesting power subsystem. In some embodiments, the patient fluid absorption and estimation device is in the form factor of a diaper or an absorbent pad. In some embodiments, sensing a voltage across each piezoresistive element comprises sampling the voltage. In some embodiments, the piezoresistive element interface comprises a resistance bridge.

These and other aspects, objects, features, and advantages of the example embodiments will become apparent to those having ordinary skill in the art upon consideration of the following summary description of illustrated example embodiments.

### BRIEF DESCRIPTION OF THE DRAWINGS

FIG. 1 is a block diagram depicting an operating environment to estimate fluid output, in accordance with certain example embodiments.

FIG. 2 is a block diagram illustrating a piezoresistive element interface, in accordance with certain example embodiments.

FIG. 3 is a notional block diagram illustrating a diaper to estimate fluid output of a patient, in accordance with certain example embodiments.

FIG. 4 is a notional block diagram illustrating a diaper to estimate fluid output of a patient, in accordance with certain example embodiments.

FIG. 5 and FIG. 6 is a block flow diagram depicting methods to estimate fluid output, in accordance with certain example embodiments.

FIG. 7 is a block diagram depicting a computing machine and a module, in accordance with certain example embodiments.

### DETAILED DESCRIPTION

Urinary output, in extreme low birth weight infants, is characterized by three phases, an oliguric phase, a polyuric phase, followed by an adaptive phase. In these most fragile infants, UO ranges from approximately 1.5-4.5 ml/kg/hr. Considering the oliguric phase (i.e., the least expected UO volume) and assuming a conservative output of 1 ml/kg/hr., and a weight of 650 grams, an expected UO at 3 hours would be 1.95 ml or approximately 2 ml of output. Thus, given a diaper change every 3 hours, and using conservative estimates of output/weight, a system sensitive enough to detect UO in whole milliliter values would be adequate for most if not all clinical situations.

Turning now to the drawings, in which like numerals represent like (but not necessarily identical) elements throughout the figures, example embodiments are described in detail.

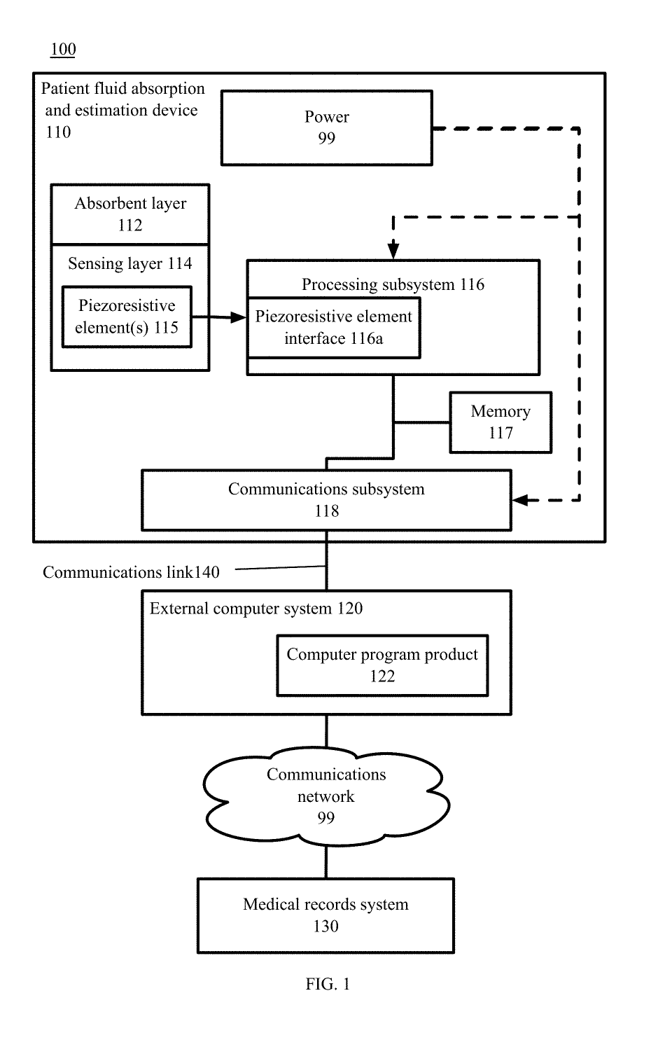

FIG. 1 is a block diagram depicting an example operating environment 100 to estimate patient fluid output, in accordance with certain example embodiments. While each server, system, and device shown in the architecture is represented by one instance of the server, system, or device, multiple instances of each can be used. Further, while certain aspects of operation of the present technology are presented in examples related to FIG. 1 to facilitate enablement of the claimed invention, additional features of the present technology, also facilitating enablement of the claimed invention, are disclosed elsewhere herein.

As depicted in FIG. 1, the operating environment 100 includes network devices 120 and 130; each of which may be configured to communicate with one another via communications network 99. The operating environment 100 also includes patient fluid absorption and estimation device 110 (hereinafter also referred to as “device 110”).

Network 99 includes one or more wired or wireless telecommunications means by which network devices may exchange data. For example, the network 99 may include one or more of a local area network (LAN), a wide area network (WAN), an intranet, an Internet, a storage area network (SAN), a personal area network (PAN), a metropolitan area network (MAN), a wireless local area network (WLAN), a virtual private network (VPN), a cellular or other mobile communication network, a BLUETOOTH® wireless technology connection, a near field communication (NFC) connection, any combination thereof, and any other appropriate architecture or system that facilitates the communication of signals, data, and/or messages. Throughout the discussion of example embodiments, it should be understood that the terms “data” and “information” are used interchangeably herein to refer to text, images, audio, video, or any other form of information that can exist in a computer-based environment.

Each network device 120 and 130 can include a communication module capable of transmitting and receiving data over the network 99. For example, each network device can include a server, a desktop computer, a laptop computer, a tablet computer, a television with one or more processors embedded therein and/or coupled thereto, a smart phone, a handheld computer, a personal digital assistant (PDA), or any other wired or wireless processor-driven device. In the example architecture depicted in FIG. 1, a hospital (or other caregiver) may operate external computer system 120, communications network 99, and medical records system 130, and may apply device 110, in the form of a diaper, to a patient.

The network connections illustrated are examples and other means of establishing a communications link between the computers and devices can be used. Moreover, those having ordinary skill in the art having the benefit of the present disclosure will appreciate that the network devices illustrated in FIG. 1 may have any of several other suitable computer system configurations. For example, external computer system 120 may be embodied as a mobile phone or handheld computer and may not include all the components described above.

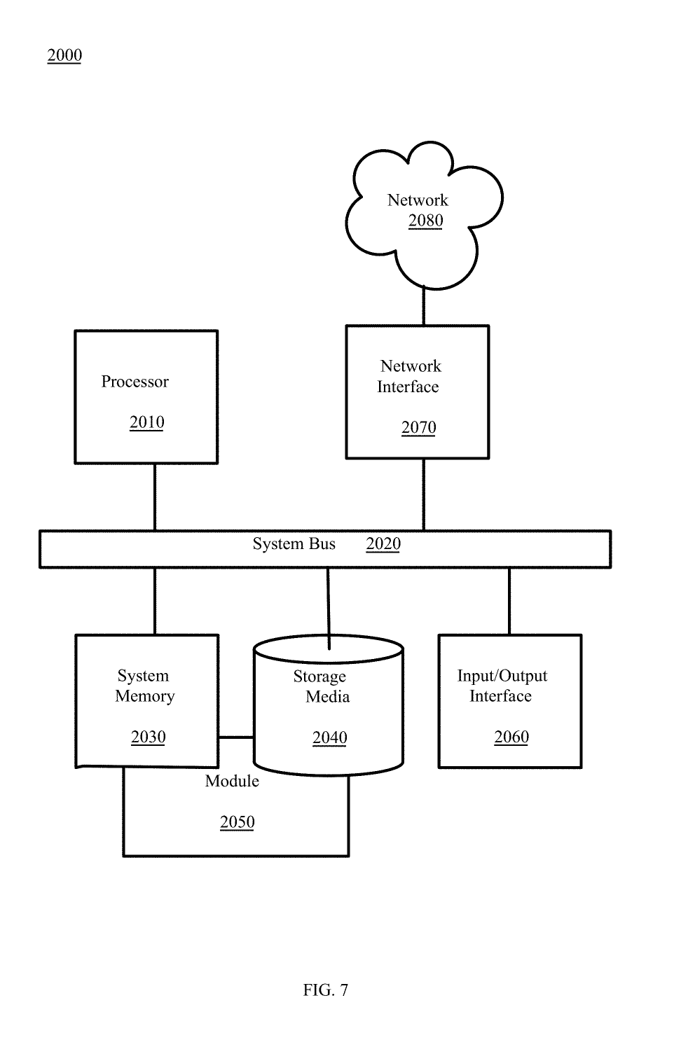

In example embodiments, the network computing devices, and any other computing machines associated with the technology presented herein, may be any type of computing machine such as, but not limited to, those discussed in more detail with respect to FIG. 7. Furthermore, any modules associated with any of these computing machines, such as modules described herein or any other modules (scripts, web content, software, firmware, or hardware) associated with the technology presented herein may be any of the modules discussed in more detail with respect to FIG. 7. The computing machines discussed herein may communicate with one another as well as other computer machines or communication systems over one or more networks, such as network 99. The network 99 may include any type of data or communications network, including any of the network technology discussed with respect to FIG. 7.

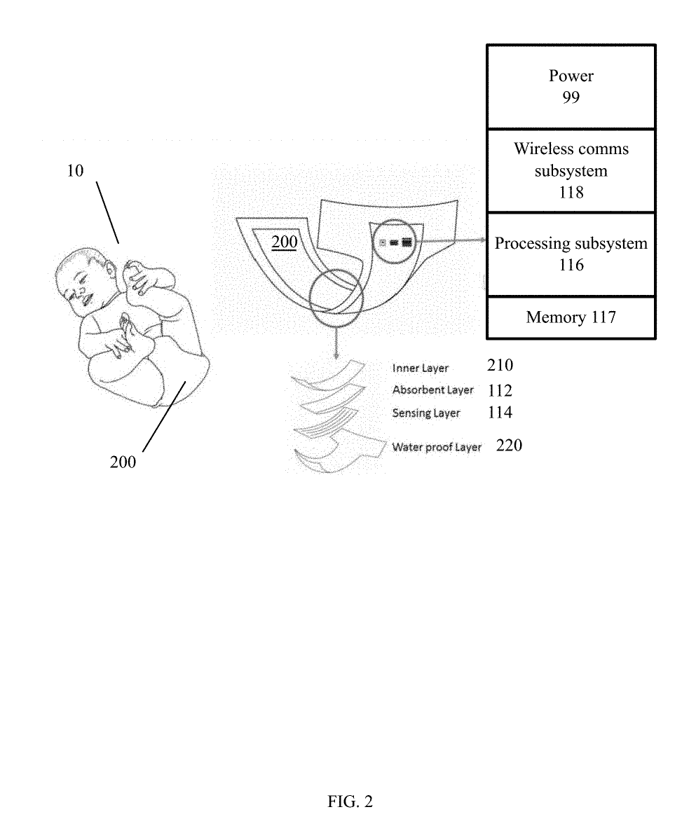

Referring to FIG. 2, and continuing to refer to FIG. 1, an example patient fluid absorption and estimation device 110 is shown as a diaper 200 that can be applied to a patient 10. In such a device 110, an inner layer 210 is a soft liner, such as a nonwoven material with a distribution layer directly beneath. The combination of nonwoven material and a distribution layer transfers fluids away from the patient 10 and toward an absorbent layer 112 to keep the patient 10 dry and comfortable.

The absorbent layer 112 includes an absorbent material adapted to absorb fluids. For example, the absorbent layer 112 can include a mixture of air-laid paper and superabsorbent polymers to absorb fluids that pass through the inner layer 210. Superabsorbent polymers (SAPs) (also called slush powder) can absorb and retain extremely large amounts of a liquid relative to their own mass. As the absorbent layer 112 absorbs fluids, it expands in both volume and mass.

The device 110 includes a sensing layer 114 comprising at least one piezoresistive element 115. The piezoresistive effect is a change in the electrical resistivity of a material when mechanical strain is applied to the material. In contrast to the piezoelectric effect, the piezoresistive effect causes a change only in electrical resistance, not in electric potential. Each piezoresistive element 115 includes a film made of one or more insulating elastomers (e.g., ethylene-co-vinyl acetate, ethylene-acrylic ester-glycidyl methacrylate, poly(vinylidene fluoride-co-hexafluoropropylene), poly(vinylidene fluoride-co-trifluoroethylene), acrylonitrile butadiene rubber) and one or more conductive fillers (e.g., carbon nanofiber, carbon black, graphite, carbon nanotube, metallic nanowire and nanoparticles), or is a nanofiber nonwoven web made of insulating elastomer and conductive fillers. The sensing layer 114 abuts the absorbent layer 112 such that changes in at least one of the mass of the absorbent layer 112 and the shape of the absorbent layer 112 cause a change in the strain in the at least one piezoresistive element 115 of the sensing layer. For example, the sensing layer 114 can be adhered to the outer surface of the absorbent layer 112 using an adhesive.

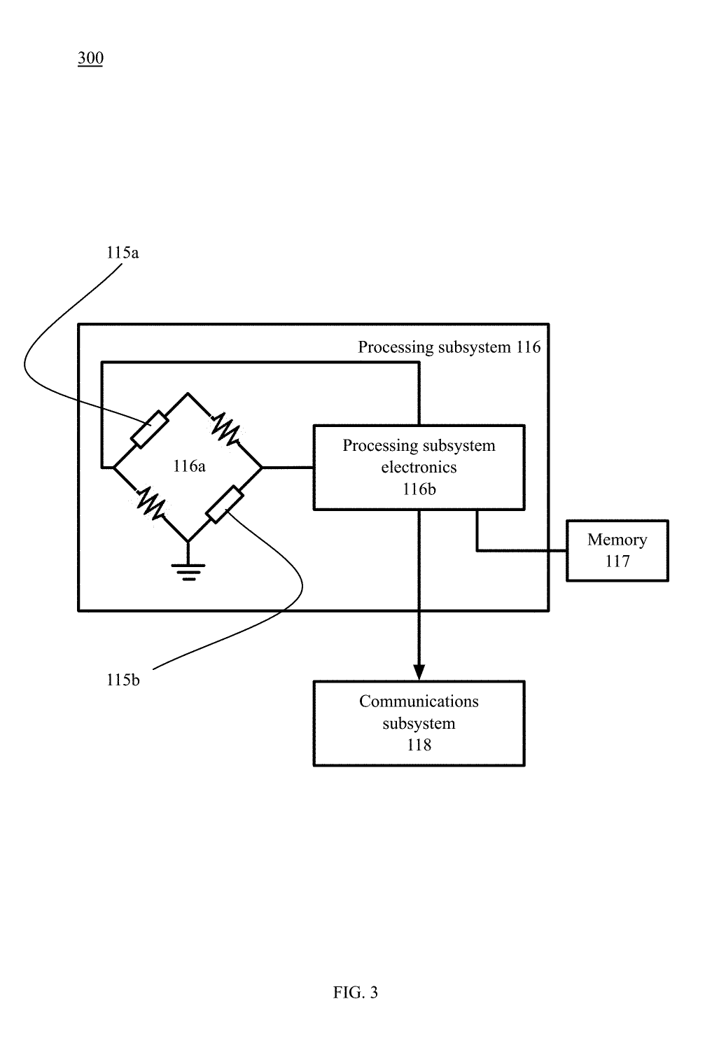

The device 110 also includes a memory 117 and a processing subsystem 116, in communication with the memory 117. The processing subsystem 116 includes a piezoresistive element interface 116a and processing subsystem electronics 116b. The processing subsystem 116 is operative to sense a voltage across each piezoresistive element 115 via the piezoresistive element interface 116a. The sensed voltage represents an estimation of the amount of fluid absorbed in the absorbent layer 112. The processing subsystem 116 converts the sensed voltage to a digital value, and stores each digital value in memory 117. As shown in FIG. 3, in some embodiments, the piezoresistive element interface 116a is a resistance bridge interfacing with two piezoresistive elements 115a, 115b as opposing sides in the four-sided bridge. In some embodiments, where a single piezoresistive element 115 is used, the remaining three resistors in the bridge are fixed value resistors. Processing subsystem electronics 116b can include a differential amplifier (for example, sensing the differential voltage across the resistance bridge 116a) and an analog-to-digital converter to convert the sensed output to a digital value to be stored in memory 117.

In some embodiments of the device 110, the processing system 116 samples the voltage across the piezoresistive elements 115 using the piezoresistive element interface 116 over time, and converts each sample to a digital value before storing each digital value in memory 117.

The device includes a communications subsystem 118, in communication with the both the processing subsystem 116 and the memory 117, and operative to transmit the stored values to an external computer system 120 via communications link 140. In some embodiments, the communications subsystem 118 is in communication with memory 117 via the processing subsystem. In some embodiments, communications subsystem 118 and communications link 140 are wireless and employ a technology such as one or more of radio frequency identification (RFID) technology, a local area network (LAN), a wide area network (WAN), an intranet, an Internet, a storage area network (SAN), a personal area network (PAN), a metropolitan area network (MAN), a wireless local area network (WLAN), a virtual private network (VPN), a cellular or other mobile communication network, a BLUETOOTH® wireless technology connection, a near field communication (NFC) connection, infrared (IR) communication, any combination thereof, and any other appropriate architecture or system that facilitates the communication of signals, data, and/or messages.

The device includes a power subsystem 99 in electrical communication with the processing subsystem 116 and the communications subsystem 118, and operative to power each subsystem.

While the example device 110 described above is in the form factor of a diaper 210, the device can take other forms, such as an absorbent pad, mat, or roll.

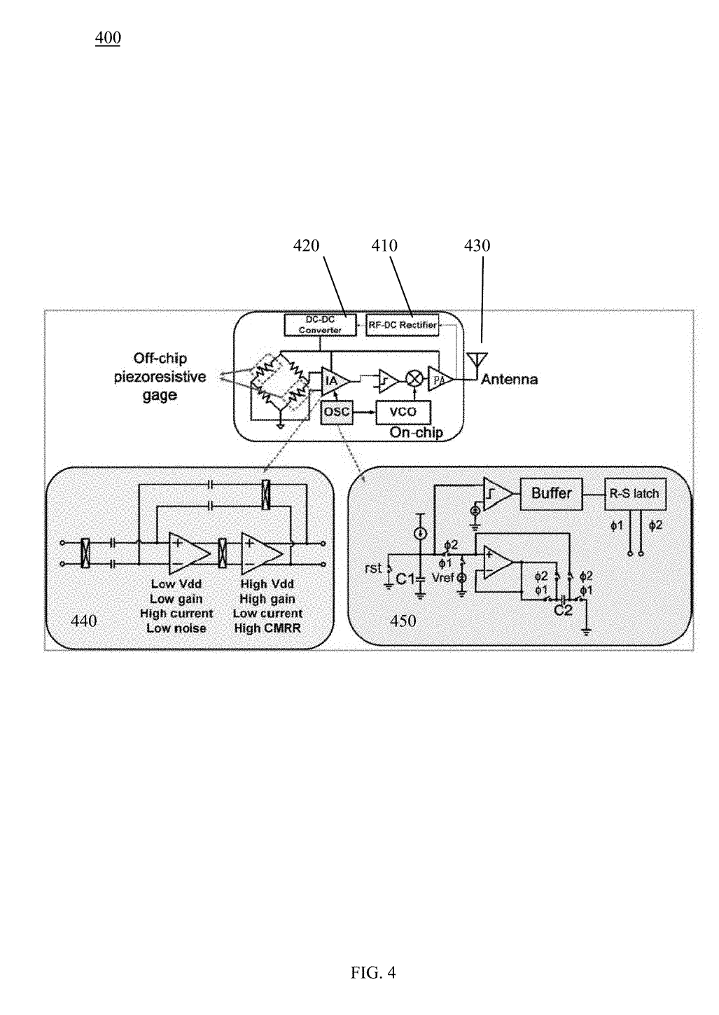

In some embodiments, power subsystem 99 includes a battery. In some embodiments, power subsystem 99 is an ambient energy harvesting subsystem, an example of which is shown in FIG. 4 as elements 410 and 420. In such embodiments, the power subsystem 99 builds on a highly sensitive analog-front-end conditioning circuit and is capable of operating with other sensors using the IEEE standard Bluetooth-low-energy protocol. FIG. 4 is a block diagram of an example analog front-end conditioning circuit with a wireless energy harvester (410 and 420) through a less than 2 cm² antenna 430. The integrated power-management unit (410 and 420) extracts energy from the RF signal in a 2-step process: RF-to-DC converter 410, DC-DC converter 420. Conventional RF-DC converters rely on off-chip inductors increasing integration costs. Some embodiments of the present technology use ultra-low cost RF-DC converter implementations reducing the integration costs to just one additional fabrication layer and eliminating the requirement of a separate inductor. This is accomplished using active devices to resonate the antenna 430 with the rectifier capacitance and using a high-storage capacitor to efficiently store the harvested energy.

Regarding the amplifiers used for the low noise differential amplifier (LNA) 440 of FIG. 3, recent techniques using chopper stabilization in low-frequency amplifiers have yielded reduced flicker noise effects resulting in the system performance being dominated by the circuit thermal noise only. In other words, the noise-efficiency (NEF) and the power efficiency factors (PEF) of the LNA 440 are limited by a direct trade-off between noise and power. Recent works have proposed efficient LNA designs. However, they are limited by the pseudo-resistor noise and lower effective transductance Gₘ at high temperatures impacting the LNA performance severely as temperature is scaled close to 80-100° C. Earlier work has shown a complementary input amplifier topology to boost the effective Gₘ and reduce the input-referred thermal noise with impressive performance. However, in order to achieve a high effective Gₘ, large current has to be drawn yielding a NEF of only 2.9. More recently, a noise-efficient inverter-based chopper amplifier with a NEF of 2.1 was demonstrated using pseudo-resistors in the common-mode feedback loop that results in a trade-off between noise and settling. Further, its pseudo-differential topology makes the common mode rejection ratio (CMRR) and power supply rejection ratio (PSRR) susceptible to process, voltage, and temperature (PVT) variations. The proposed digital calibration technique overcomes the noise limitation across wide-range of temperatures. The amplifier achieves one of the best noise and power efficiencies and a stable low noise performance across a wide range of temperature and process corners. The standby mode uses the outcome of the feature-extractor to realize even higher power-efficiencies yet maintaining faster start-up times.

Regarding the reference oscillator 450, relaxation oscillators are widely used as on-chip reference for low frequency applications especially in biomedical sensor nodes because of the low power operation and on-chip integration. However, the oscillating frequency has shown to be very sensitive to PVT variation limiting phase noise achieved. This work adopts a switched-current technique proposed earlier for an 11 Hz oscillator out uses only one opamp while still maintaining a good frequency stability and low phase noise. The relaxation oscillator will also be used as a wake-up timer that provides the onset signal for the whole system. By combining the analog feature extraction, a closed loop control system will be formed to power ON/OFF the system and thus improve the overall system power efficiency.

In some embodiments, the present technology is embodied in a system including the device 110 as described above, and a computer program product 122. The computer program product 122 is non-transitory computer-readable storage device having computer-executable program instructions embodied thereon. The computer-readable storage device can be a memory, including a portable media, such as a compact disc (CD) or flash drive, that is not part of the external computer system 120. However, when the computer-executable program instructions of the computer program product 122 are installed in the external computer system 120, the memory used by the external computer system 120 becomes part of the computer program product 122. When the instructions are executed by the external computer system 120, they cause the external computer system 120 to interface with the device 110. The computer-executable program instructions include instructions to command the device 110, via the external computer system 120 and over communications link 140, to transmit the stored values from memory 117 in the device 110 to the external computer system 120. The computer-executable program instructions include instructions to receive, in response to the command, and from the device 110 via into the external computer system 120, the transmitted values. The computer-executable program instructions include instructions to convert the received values to an estimate of fluid absorbed by the device 110.

In some embodiments, the computer-executable program instructions further include instructions to transmit the converted values from the external computer system 120 to a medical records system 130, as an estimate of fluid absorbed by the device.

The example devices and methods illustrated in the figures are described hereinafter with respect to the components of the example operating environment 100. The example methods also can be performed with other systems and in other environments. The operations described with respect to any of the figures can be implemented as executable code stored on a computer or machine readable non-transitory tangible storage medium (e.g., floppy disk, hard disk, ROM, EEPROM, nonvolatile RAM, CD-ROM, etc.) that are completed based on execution of the code by a processor circuit implemented using one or more integrated circuits; the operations described herein also can be implemented as executable logic that is encoded in one or more non-transitory tangible media for execution (e.g., programmable logic arrays or devices, field programmable gate arrays, programmable array logic, application specific integrated circuits, etc.).

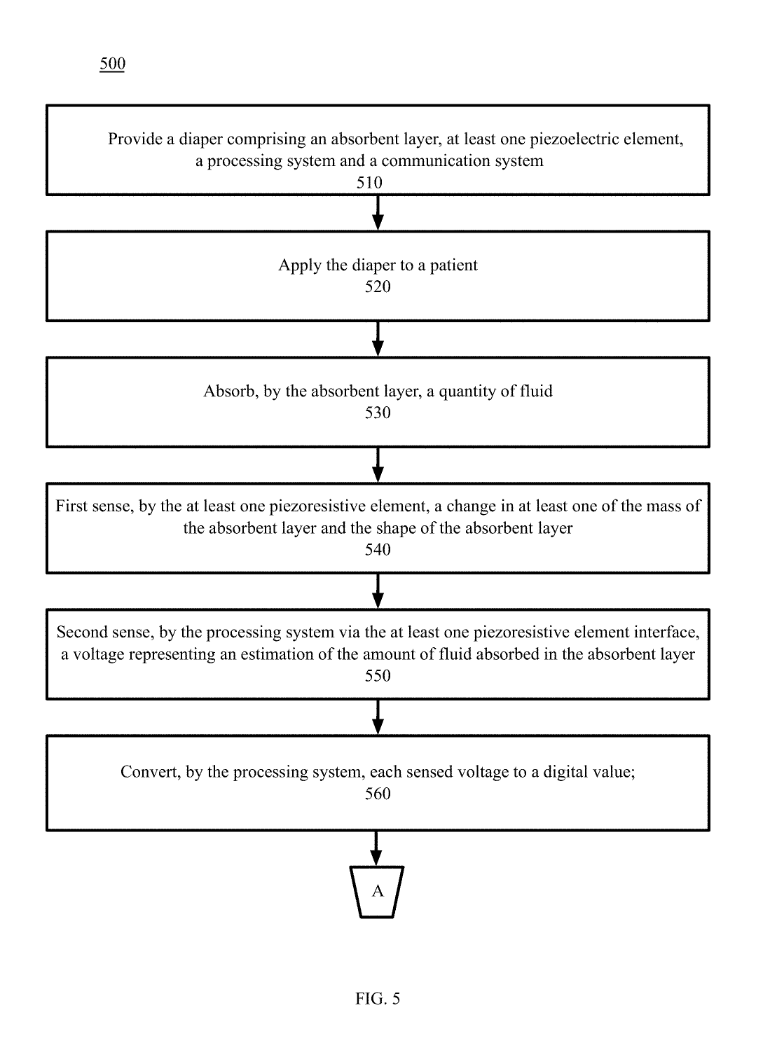

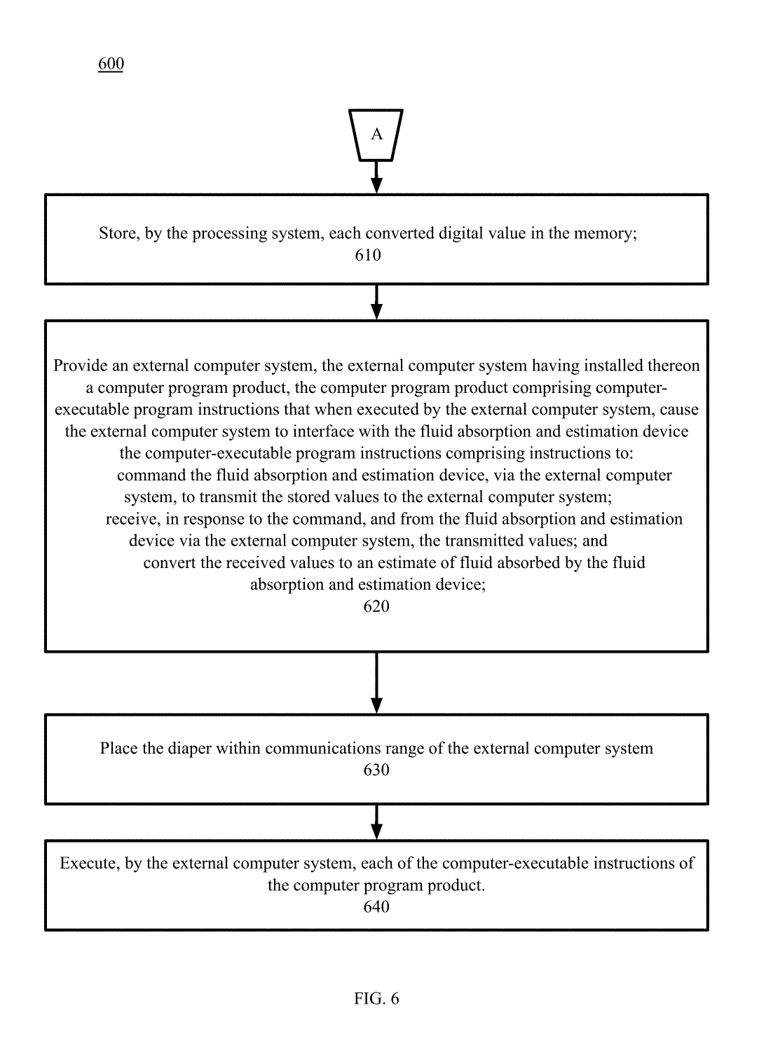

Referring to FIG. 5, and continuing to refer to prior figures for context, a block flow diagram depicting methods 500 to estimate patient fluid output in accordance with certain example embodiments is shown.

In such methods 500, a patient fluid absorption and estimation device 110 is provided—Block 510. The device 110 is structured and operates as described above using a single 3 cm×2 cm piezoresistive element 115. The device 110 is applied to a patient 10—Block 520. The absorbent layer 112 absorbs a quantity of fluid—Block 530. In the continuing example, the absorbent layer absorbs a quantity of fluid in the range of 1-15 ml.

The at least one piezoresistive element 115 first senses a change in at least one of the mass of the absorbent layer 112 and the shape of the absorbent layer 112; the first sensing causing a change in the strain in the at least one piezoresistive element 115—Block 540. In the continuing example, the 0-15 ml of fluid absorbed by the absorbent layer 112 creates a pressure change on the piezoresistive element 115 in the range of 0-100 Pa. Specifically, the absorbent layer 112 absorbs 2 ml in the continuing example.

The processing subsystem 116 second senses, via the at least one piezoresistive element interface 116a, a voltage representing an estimation of the amount of fluid absorbed in the absorbent layer 112—Block 550. The processing subsystem 116, specifically the processing subsystem electronics 116b, converts each sensed voltage to a digital value—Block 560. In the continuing example, a 6-bit analog-to-digital converter provides 64 separate levels over the 0-15 ml range—offering a resolution of at least 0.25 ml. The processing system 116 stores each converted digital value in the memory 117—Block 610. In the continuing example, a bit value of 000100 is stored in memory, corresponding to the voltage output in response to strain induced in the piezoresistive element by the 2 ml of fluid absorbed in the absorbent layer.

An external computer system 120 is provided having installed thereon a computer program product 122—Block 620. As described above, the computer program product 122 includes computer-executable program instructions that when executed by the external computer system 120, cause the external computer system 120 to interface with the fluid absorption and estimation device 110.

The device 110 is placed within communications range of the external computer system 120—Block 630. The external computer system 120, executes each of the computer-executable instructions of the computer program product 122—Block 640. In the continuing example, a bit value of 000100 using 0.25 ml resolution from 0 ml corresponds to 2 ml.

### OTHER EXAMPLE EMBODIMENTS

FIG. 7 depicts a computing machine 2000 and a module 2050 in accordance with certain example embodiments. The computing machine 2000 may correspond to any of the various computers, servers, mobile devices, embedded systems, or computing systems presented herein. The module 2050 may comprise one or more hardware or software elements configured to facilitate the computing machine 2000 in performing the various methods and processing functions presented herein. The computing machine 2000 may include various internal or attached components such as a processor 2010, system bus 2020, system memory 2030, storage media 2040, input/output interface 2060, and a network interface 2070 for communicating with a network 2080.

The computing machine 2000 may be implemented as a conventional computer system, an embedded controller, a laptop, a server, a mobile device, a smartphone, a set-top box, a kiosk, a router or other network node, a vehicular information system, one or more processors associated with a television, a customized machine, any other hardware platform, or any combination or multiplicity thereof. The computing machine 2000 may be a distributed system configured to function using multiple computing machines interconnected via a data network or bus system.

The processor 2010 may be configured to execute code or instructions to perform the operations and functionality described herein, manage request flow and address mappings, and to perform calculations and generate commands. The processor 2010 may be configured to monitor and control the operation of the components in the computing machine 2000. The processor 2010 may be a general purpose processor, a processor core, a multiprocessor, a reconfigurable processor, a microcontroller, a digital signal processor (“DSP”), an application specific integrated circuit (“ASIC”), a graphics processing unit (“GPU”), a field programmable gate array (“FPGA”), a programmable logic device (“PLD”), a controller, a state machine, gated logic, discrete hardware components, any other processing unit, or any combination or multiplicity thereof. The processor 2010 may be a single processing unit, multiple processing units, a single processing core, multiple processing cores, special purpose processing cores, co-processors, or any combination thereof. According to certain embodiments, the processor 2010 along with other components of the computing machine 2000 may be a virtualized computing machine executing within one or more other computing machines.

The system memory 2030 may include non-volatile memories such as read-only memory (“ROM”), programmable read-only memory (“PROM”), erasable programmable read-only memory (“EPROM”), flash memory, or any other device capable of storing program instructions or data with or without applied power. The system memory 2030 may also include volatile memories such as random access memory (“RAM”), static random access memory (“SRAM”), dynamic random access memory (“DRAM”), and synchronous dynamic random access memory (“SDRAM”). Other types of RAM also may be used to implement the system memory 2030. The system memory 2030 may be implemented using a single memory module or multiple memory modules. While the system memory 2030 is depicted as being part of the computing machine 2000, one skilled in the art will recognize that the system memory 2030 may be separate from the computing machine 2000 without departing from the scope of the subject technology. It should also be appreciated that the system memory 2030 may include, or operate in conjunction with, a non-volatile storage device such as the storage media 2040.

The storage media 2040 may include a hard disk, a floppy disk, a compact disc read only memory (“CD-ROM”), a digital versatile disc (“DVD”), a Blu-ray disc, a magnetic tape, a flash memory, other non-volatile memory device, a solid state drive (“SSD”), any magnetic storage device, any optical storage device, any electrical storage device, any semiconductor storage device, any physical-based storage device, any other data storage device, or any combination or multiplicity thereof. The storage media 2040 may store one or more operating systems, application programs and program modules such as module 2050, data, or any other information. The storage media 2040 may be part of, or connected to, the computing machine 2000. The storage media 2040 may also be part of one or more other computing machines that are in communication with the computing machine 2000 such as servers, database servers, cloud storage, network attached storage, and so forth.

The module 2050 may comprise one or more hardware or software elements configured to facilitate the computing machine 2000 with performing the various methods and processing functions presented herein. The module 2050 may include one or more sequences of instructions stored as software or firmware in association with the system memory 2030, the storage media 2040, or both. The storage media 2040 may therefore represent examples of machine or computer readable media on which instructions or code may be stored for execution by the processor 2010. Machine or computer readable media may generally refer to any medium or media used to provide instructions to the processor 2010. Such machine or computer readable media associated with the module 2050 may comprise a computer software product. It should be appreciated that a computer software product comprising the module 2050 may also be associated with one or more processes or methods for delivering the module 2050 to the computing machine 2000 via the network 2080, any signal-bearing medium, or any other communication or delivery technology. The module 2050 may also comprise hardware circuits or information for configuring hardware circuits such as microcode or configuration information for an FPGA or other PLD.

The input/output (“I/O”) interface 2060 may be configured to couple to one or more external devices, to receive data from the one or more external devices, and to send data to the one or more external devices. Such external devices along with the various internal devices may also be known as peripheral devices. The I/O interface 2060 may include both electrical and physical connections for operably coupling the various peripheral devices to the computing machine 2000 or the processor 2010. The I/O interface 2060 may be configured to communicate data, addresses, and control signals between the peripheral devices, the computing machine 2000, or the processor 2010. The I/O interface 2060 may be configured to implement any standard interface, such as small computer system interface (“SCSI”), serial-attached SCSI (“SAS”), fiber channel, peripheral component interconnect (“PCP”), PCI express (PCIe), serial bus, parallel bus, advanced technology attached (“ATA”), serial ATA (“SATA”), universal serial bus (“USB”), Thunderbolt, FireWire, various video buses, and the like. The I/O interface 2060 may be configured to implement only one interface or bus technology. Alternatively, the I/O interface 2060 may be configured to implement multiple interfaces or bus technologies. The I/O interface 2060 may be configured as part of, all of, or to operate in conjunction with, the system bus 2020. The I/O interface 2060 may include one or more buffers for buffering transmissions between one or more external devices, internal devices, the computing machine 2000, or the processor 2010.

The I/O interface 2060 may couple the computing machine 2000 to various input devices including mice, touch-screens, scanners, electronic digitizers, sensors, receivers, touchpads, trackballs, cameras, microphones, keyboards, any other pointing devices, or any combinations thereof. The I/O interface 2060 may couple the computing machine 2000 to various output devices including video displays, speakers, printers, projectors, tactile feedback devices, automation control, robotic components, actuators, motors, fans, solenoids, valves, pumps, transmitters, signal emitters, lights, and so forth.

The computing machine 2000 may operate in a networked environment using logical connections through the network interface 2070 to one or more other systems or computing machines across the network 2080. The network 2080 may include wide area networks (WAN), local area networks (LAN), intranets, the Internet, wireless access networks, wired networks, mobile networks, telephone networks, optical networks, or combinations thereof. The network 2080 may be packet switched, circuit switched, of any topology, and may use any communication protocol. Communication links within the network 2080 may involve various digital or an analog communication media such as fiber optic cables, free-space optics, waveguides, electrical conductors, wireless links, antennas, radio-frequency communications, and so forth.

The processor 2010 may be connected to the other elements of the computing machine 2000 or the various peripherals discussed herein through the system bus 2020. It should be appreciated that the system bus 2020 may be within the processor 2010, outside the processor 2010, or both. According to certain example embodiments, any of the processor 2010, the other elements of the computing machine 2000, or the various peripherals discussed herein may be integrated into a single device such as a system on chip (“SOC”), system on package (“SOP”), or ASIC device.

Embodiments may comprise a computer program that embodies the functions described and illustrated herein, wherein the computer program is implemented in a computer system that comprises instructions stored in a machine-readable medium and a processor that executes the instructions. However, it should be apparent that there could be many different ways of implementing embodiments in computer programming, and the embodiments should not be construed as limited to any one set of computer program instructions. Further, a skilled programmer would be able to write such a computer program to implement an embodiment of the disclosed embodiments based on the appended flow charts and associated description in the application text. Therefore, disclosure of a particular set of program code instructions is not considered necessary for an adequate understanding of how to make and use embodiments. Further, those skilled in the art will appreciate that one or more aspects of embodiments described herein may be performed by hardware, software, or a combination thereof, as may be embodied in one or more computing systems. Moreover, any reference to an act being performed by a computer should not be construed as being performed by a single computer as more than one computer may perform the act.

The example embodiments described herein can be used with computer hardware and software that perform the methods and processing functions described herein. The systems, methods, and procedures described herein can be embodied in a programmable computer, computer-executable software, or digital circuitry. The software can be stored on computer-readable media. For example, computer-readable media can include a floppy disk, RAM, ROM, hard disk, removable media, flash memory, memory stick, optical media, magneto-optical media, CD-ROM, etc. Digital circuitry can include integrated circuits, gate arrays, building block logic, field programmable gate arrays (FPGA), etc.

The example systems, methods, and acts described in the embodiments presented previously are illustrative, and, in alternative embodiments, certain acts can be performed in a different order, in parallel with one another, omitted entirely, and/or combined between different example embodiments, and/or certain additional acts can be performed, without departing from the scope and spirit of various embodiments. Accordingly, such alternative embodiments are included in the scope of the following claims, which are to be accorded the broadest interpretation to encompass such alternate embodiments.

Although specific embodiments have been described above in detail, the description is merely for purposes of illustration. It should be appreciated, therefore, that many aspects described above are not intended as required or essential elements unless explicitly stated otherwise. Modifications of, and equivalent components or acts corresponding to, the disclosed aspects of the example embodiments, in addition to those described above, can be made by a person of ordinary skill in the art, having the benefit of the present disclosure, without departing from the spirit and scope of embodiments defined in the following claims, the scope of which is to be accorded the broadest interpretation so as to encompass such modifications and equivalent structures.

1. A patient fluid absorption and estimation device, comprising:
an absorbent layer comprising an absorbent material and adapted to absorb fluids;
a sensing layer:
comprising at least one piezoresistive element, and
abutting the absorbent layer such that changes in at least one of the mass of the absorbent layer and the shape of the absorbent layer cause a change in the strain in the at least one piezoresistive element;
a memory;
a processing subsystem, in communication with the memory:
the processing subsystem comprising a piezoresistive element interface; and
the processing subsystem operative to:
sense a voltage across each piezoresistive element via the piezoresistive element interface, the sensed voltage representing an estimation of the amount of fluid absorbed in the absorbent layer;
convert each sensed voltage to a digital value; and
store each digital value in the memory;
a wireless communications subsystem, in communication with the processing subsystem and the memory, and operative to wirelessly transmit the stored values to an external device; and
a power subsystem in electrical communication with the processing subsystem and the communications subsystem, and operative to power each subsystem.

2. The device of claim 1, wherein the power subsystem comprises an ambient harvesting power subsystem.

3. The device of claim 1, wherein the patient fluid absorption and estimation device is in the form factor of a diaper.

4. The device of claim 1, wherein the patient fluid absorption and estimation device is in the form factor of an absorbent pad.

5. The device of claim 1, wherein sensing a voltage across each piezoresistive element comprises sampling the voltage.

6. The device of claim 1, wherein the piezoresistive element interface comprises a resistance bridge.

7. A fluid absorption and estimation system, comprising:
a fluid absorption and estimation device, comprising:
an absorbent layer comprising an absorbent material and adapted to absorb fluids;
a sensing layer comprising at least one piezoresistive element, and abutting the absorbent layer such that changes in at least one of the mass of the absorbent layer and the shape of the absorbent layer cause a change in the strain in the at least one piezoresistive element;
a memory;
a processing subsystem, in communication with the memory and comprising a piezoresistive element interface; the processing subsystem operative to sense a voltage, representing an estimation of the amount of fluid absorbed in the absorbent layer, across each piezoresistive element via the piezoresistive element interface, to convert each sensed voltage to a digital value, and to store each digital value in the memory;
a wireless communications subsystem, in communication with the processing subsystem and the memory, and operative to receive commands from an external computer system to transmit the stored values, and operative to wirelessly transmit the stored values to the external computer system in response to the commands; and
a power subsystem in electrical communication with the processing subsystem and the communications subsystem, and operative to power each subsystem; and
a non-transitory computer-readable storage device having computer-executable program instructions embodied thereon that when executed by the external computer system, cause the external computer system to interface with the fluid absorption and estimation device, the computer-executable program instructions comprising:
computer-executable program instructions to command the fluid absorption and estimation device, via the external computer system, to transmit the stored values to the external computer system;
computer-executable program instructions to receive, in response to the command, and from the fluid absorption and estimation device via the external computer system, the transmitted values; and
computer-executable program instructions to convert the received values to an estimate of fluid absorbed by the fluid absorption and estimation device.

8. The system of claim 7, wherein the computer-executable program instructions further comprise:
computer-executable program instructions to transmit the converted values from the external computer system to a medical records system, as an estimate of fluid absorbed by the device.

9. The system of claim 7, wherein the power subsystem comprises an ambient harvesting power subsystem.

10. The system of claim 7, wherein the patient fluid absorption and estimation device is in the form factor of a diaper.

11. The system of claim 7, wherein the patient fluid absorption and estimation device is in the form factor of an absorbent pad.

12. The system of claim 7, wherein sensing a voltage across each piezoresistive element comprises sampling the voltage.

13. The device of claim 7, wherein the piezoresistive element interface comprises a resistance bridge.

14. A method to estimate patient fluid output, comprising:
providing a patient fluid absorption and estimation device, comprising:
an absorbent layer comprising an absorbent material and adapted to absorb fluids;
a sensing layer comprising at least one piezoresistive element, and abutting the absorbent layer such that changes in at least one of the mass of the absorbent layer and the shape of the absorbent layer cause a change in the strain in the at least one piezoresistive element;
a memory;
a processing subsystem, in communication with the memory and comprising a piezoresistive element interface; the processing subsystem operative to sense a voltage, representing an estimation of the amount of fluid absorbed in the absorbent layer, across each piezoresistive element via the piezoresistive element interface, to convert each sensed voltage to a digital value, and to store each digital value in the memory;
a wireless communications subsystem, in communication with the processing subsystem and the memory, and operative to receive commands from an external computer system to transmit the stored values, and operative to wirelessly transmit the stored values to the external computer system in response to the commands; and
a power subsystem in electrical communication with the processing subsystem and the communications subsystem, and operative to power each subsystem;
applying the patient fluid absorption and estimation device to a patient;
absorbing, by the absorbent layer, a quantity of fluid;
first sensing, by the at least one piezoresistive element, a change in at least one of the mass of the absorbent layer and the shape of the absorbent layer, the first sensing causing a change in the strain in the at least one piezoresistive element;
second sensing, by the processing system via the at least one piezoresistive element interface, a voltage representing an estimation of the amount of fluid absorbed in the absorbent layer;
converting, by the processing system, each sensed voltage to a digital value;
storing, by the processing system, each converted digital value in the memory;
providing an external computer system, the external computer system having installed thereon a computer program product, the computer program product comprising computer-executable program instructions that when executed by the external computer system, cause the external computer system to interface with the fluid absorption and estimation device, the computer-executable program instructions comprising:
computer-executable program instructions to command the fluid absorption and estimation device, via the external computer system, to transmit the stored values to the external computer system;
computer-executable program instructions to receive, in response to the command, and from the fluid absorption and estimation device via the external computer system, the transmitted values; and
computer-executable program instructions to convert the received values to an estimate of fluid absorbed by the fluid absorption and estimation device;
placing the diaper within communications range of the external computer system; and
executing, by the external computer system, each of the computer-executable instructions of the computer program product.

15. The method of claim 14:
wherein the computer-executable program instructions further comprise computer-executable program instructions to transmit the converted values from the external computer system to a medical records system, as an estimate of fluid absorbed by the device; and
wherein the method further comprises transmitting, by the external computer system in accordance with the instructions, the converted values from the external computer system to a medical records system, as an estimate of fluid absorbed by the device.

16. The method of claim 14, wherein the power subsystem comprises an ambient harvesting power subsystem.

17. The method of claim 14, wherein the patient fluid absorption and estimation device is in the form factor of a diaper.

18. The method of claim 14, wherein the patient fluid absorption and estimation device is in the form factor of an absorbent pad.

19. The method of claim 14, wherein sensing a voltage across each piezoresistive element comprises sampling the voltage.

20. The method of claim 14, wherein the piezoresistive element interface comprises a resistance bridge.
